(19) United States
(12) Patent Application Publication
(10) Pub. No.: US 2020/0396985 A1
(43) Pub. Date: Dec. 24, 2020

(54) PRESERVATION AND CRYOPRESERVATION MEDIA

(71) Applicant: Akron Biotechnology, LLC, Boca Raton, FL (US)

(72) Inventor: Claudia Zylberberg, Delray Beach, FL (US)

(21) Appl. No.: 16/968,414

(22) PCT Filed: Feb. 11, 2019
(86) PCT No.: PCT/US2019/017488

§ 371 Date: Aug. 7, 2020

Related U.S. Application Data
(60) Provisional application No. 62/637,030, filed on Mar. 1, 2018.
(60) Provisional application No. 62/628,387, filed on Feb. 9, 2018.

(51) Int. Cl. A01N 1/02; A61K 31/505; A61K 47/26; A61K 47/18

(52) U.S. Cl. CPC A01N 1/0221; A01N 1/0226; A61K 47/18; A61K 47/26; A61K 31/505

(57) ABSTRACT

Cryopreservation media which include additives for cryopreservation of biological samples which maintain viability even after multiple freeze-thaw cycles.

### CROSS REFERENCE TO RELATED APPLICATIONS

This Application claims the benefit of U.S. Provisional Application 62/628,387 filed on Feb. 9, 2018 and U.S. Provisional Application 62/637,030 filed on Mar. 1, 2019. The entire contents of these applications are incorporated herein by reference in their entirety.

### FIELD OF THE INVENTION

Embodiments of the invention are directed to compositions suitable for culturing, preservation or cryopreservation of biological samples which maintain the viability of these biological samples even after multiple freeze-thaw cycles. In particular, the compositions include polymers, polysaccharides, carboxylated-polyamino acids, polyamino acids and other organic or inorganic molecules whether synthetic or natural.

### BACKGROUND

Cryopreservation is a process that preserves organelles, cells, tissues, or any other biological constructs by cooling the samples to very low temperatures. The responses of living cells to ice formation are of theoretical interest and practical relevance. Stem cells and other viable tissues, which have great potential for use in basic research as well as for many medical applications, cannot be stored with simple cooling or freezing for a long time because ice crystal formation, osmotic shock, and membrane damage during freezing and thawing will cause cell death.

Biological and chemical reactions in living cells are dramatically reduced at low temperature, a phenomenon that can lead to the possible long-term preservation of cells and tissues. However, freezing is fatal to most living organisms, since both intra- and extracellular ice crystals are formed and results in changes to the chemical setting of cells that lead to cellular mechanical constraints and injury. The major hurdle for cells to overcome at low temperatures is the water-to-ice phase transition. Cell injury at fast cooling rates is attributed to intracellular ice formation, whereas slow cooling causes osmotic changes due to the effects of exposure to highly concentrated intra- and extracellular solutions or to mechanical interactions between cells and the extracellular ice.

### SUMMARY

Embodiments of the invention are directed to cryopreservation media, for the freezing of biological samples, e.g. tissues, cells, without damaging of the samples even after repeated freeze-that cycles, preservation media and culture media for biological samples.

Other aspects are described infra.

### BRIEF DESCRIPTION OF THE DRAWINGS

FIG. 1. Post-thaw viability of cells cryopreserved in CP FORMULATION. Cells were frozen at a concentration of 10⁵-10⁶ cells/mL. Cells were stored at −80° C. for 24 hours and then transferred to liquid nitrogen (<−135° C.). After storage in liquid nitrogen for at least 3 days, cells were thawed, and post-thaw viability was assessed. Cells were allowed to recover for at least an additional two days and no changes in cell morphology were observed.

FIG. 2 shows a comparison of post-thaw % recovery of MCS (experimental set 1).

FIGS. 3A-3C show the post-thaw MSC morphology 72 h after plating. FIG. 3A. Control: 90% PBS +10% DMSO. FIG. 3B. CP FORMULATION A. FIG. 3C. CP FORMULATION B.

FIG. 4 is a graph showing Experimental set 2 comparison of post-thaw % recovery of mesenchymal set cells (MSCs).

FIGS. 5A-5C show the post-thaw MSC morphology 72 h after plating. FIG. 5A. Control: 90% FBS+10% DMSO. FIG. 5B. CP FORMULATION #2. FIG. 5C. CP FORMULATION #3.

FIG. 6 is a graph showing the post-thaw cell counts.

FIGS. 7A-7D show the comparison of post-thaw morphology of (FIG. 7A) DMSO+FBS; (FIG. 7B) CP FORMULATION Run #1; (FIG. 7C) CP FORMULATION Run #2; (FIG. 7D) CP FORMULATION Run #3.

FIGS. 8A-8C: Bioinspired biocompatible cryoprotectants for cryopreservation of natural killer cells. FIG. 8A) Schematic showing the chemical structures of dextran and carboxylated poly-L-lysine (CPLL). FIG. 8B) Schematic demonstrating the potential mechanism of action of dextran and CPLL during cryopreservation of natural killer (NK) cells. The synergic effect of CPAs is related to their high affinity to cell membrane, water molecules, and solutes. This characteristic might provide cell protection while removing intracellular water, restricting solute diffusion, and controlling the degree of dehydration to a level sufficient to minimize intracellular ice formation during cooling. Carboxylated PLL also might limit cryoinjury to cells by binding to ice crystals and inhibiting their growth and recrystallization during rewarming. FIG. 8C) Determination of percentage (%) cell viability following CPA loading and unloading. Low level (i.e., 5% w/v) of dextran/CPLL-based cocktail solution was used for subsequent experiments since there is no significant difference in cell viability between 5 and 10% dextran concentrations. The data shown are averages with standard error of the mean (SEM) from various independent experiments. For 5% dextran/CPLL group Nₑₓₚₑᵣᵢₘₑₙₜₛ=3; nₜₒₜₐₗ cells=315 and for 10% dextran/CPLL Nₑₓₚₑᵣᵢₘₑₙₜₛ=3; nₜₒₜₐₗ cells=416.

FIGS. 9A-9D: Assessment of NK cell viability following dextran/carboxylated poly-L-lysine (CPLL) based cryopreservation and rewarming. FIG. 9A) Schematic showing the cryopreservation protocol used for preservation of natural killer (NK) cells. The concentrated NK cells are loaded with bioinspired dextran/CPLL-based cryoprotective agent (CPA) at room temperature (24° C.). The cells are subsequently placed into cryovials, cryopreserved using slow freezing method at −80° C. The cells were then stored for 1 week. Following rapid rewarming at 37° C., the CPAs washed out from the cells by re-suspending the cells in NK media. FIGS. 9B, 9C) Determination of percentage (%) cell viability following FIG. 9B) CPA loading and unloading; FIG. 9C) cryopreservation, rewarming, and washing the CPAs; and FIG. 9D) in culture for up to 1 week. The data shown are averages with standard error of the mean (SEM) from various independent experiments. For CPA loading and unloading experiments: (i) cell medium group (Nₑₓₚₑᵣᵢₘₑₙₜₛ=7; nₜₒₜₐₗ cells=1316), (ii) DMSO group (Nₑₓₚₑᵣᵢₘₑₙₜₛ=6; nₜₒₜₐₗ cells=877), and iii) dextran/CPLL group (Nₑₓₚₑᵣᵢₘₑₙₜₛ=3 nₜₒₜₐₗ cells=315). For cryopreservation experiments: (i) DMSO group (Nₑₓₚₑᵣᵢₘₑₙₜₛ=3; nₜₒₜₐₗ cells=654) and (ii) dextran/CPLL group (Nₑₓₚₑᵣᵢₘₑₙₜₛ=3; nₜₒₜₐₗ cells=281). For 1 d in culture experiments: (i) cell medium group (Nₑₓₚₑᵣᵢₘₑₙₜₛ=3; nₜₒₜₐₗ cells=1152), (ii) DMSO group (Nₑₓₚₑᵣᵢₘₑₙₜₛ=3 nₜₒₜₐₗ cells=772), and (iii) dextran/CPLL group (Nₑₓₚₑᵣᵢₘₑₙₜₛ=3 nₜₒₜₐₗ cells=416). For 1 week in culture experiments: (i) Cell medium group (Nₑₓₚₑᵣᵢₘₑₙₜₛ=3; nₜₒₜₐₗ cells=3353), (ii) DMSO group (Nₑₓₚₑᵣᵢₘₑₙₜₛ=3; nₜₒₜₐₗ cells=3657), and (iii) dextran/CPLL group (Nₑₓₚₑᵣᵢₘₑₙₜₛ=3; nₜₒₜₐₗ cells=2219).

FIGS. 10A-10B: Assessment of NK cell functionality following dextran/carboxylated poly-L-lysine (CPLL) based cryopreservation and rewarming. Anti-tumor functional activity of recovered NK cells after dextran/CPLL- and DMSO-based solutions was evaluated against K562 leukemia cell line using cytotoxicity assay. Two different effector cells: target cells ratios were assessed (i.e., 5:1 (50 000:10 000) and 10:1 (100 000:10 000)). FIG. 10A) Representative flow cytometry dot plots. FIG. 10B) Quantification of flow cytometry analysis. The data shown are averages with standard error of the mean (SEM) from various independent experiments (n=3-4).

### DETAILED DESCRIPTION

Unless otherwise defined, all terms (including technical and scientific terms) used herein have the same meaning as commonly understood by one of ordinary skill in the art to which this disclosure pertains. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The terminology used herein is for the purpose of describing particular embodiments only and is not intended to be limiting of the disclosure. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used in this specification and the appended claims, the term “or” is generally employed in its sense including “and/or” unless the content clearly dictates otherwise.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude, preferably within 5-fold, and also preferably within 2-fold, of a value. Where particular values are described in the application and claims, unless otherwise stated the term “about” meaning within an acceptable error range for the particular value should be assumed.

An “amphoteric” agent is one which can act either as an acid or base depending on the reaction in which it is involved. “Organic amphoteric agents” are organic molecules that contain both acidic (e.g., carboxyl) and basic (e g , amino) functional groups. Thus, for example, an organic amphoteric agent includes an amino group (NH₂) and a carboxylic group (COOH) bound to the same or different carbon atoms of a hydrocarbonic backbone. Further functional groups include, for example, an amino group (NH2), carboxylic group (COOH), carbonyl group (CO), hydroxy (OH) or mercapto group (SH) or aryls like phenyl. In embodiments, the organic amphoteric agent may be ectoine, hydroxyectoine, ectoine derivatives, hydroxyectoine derivatives, analogs, variants or combinations thereof. In some embodiments the ectoine derivatives comprise acetylhydroxectoine, hydroxyectoine, homoectoin, stearoylhydroxyectoine, myristylectoin, or combinations thereof. In some embodiments, the organic amphoteric agent is ectoine and/or hydroxyectoine.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

A “biological medium” as used herein, is any type of medium that is used to grow, culture and maintain organs, tissues, cells etc., in vitro. A biological medium also encompasses any biocompatible agent, any pharmaceutical excipient, pharmaceutically and physiologically acceptable fluids such as water, physiological saline, balanced salt solutions, aqueous dextrose, glycerol or the like as a vehicle, tissue or organ culture media, any agent that can be administered in vivo to a subject, any agent that can be used in assays or for diluting or maintaining a biological sample, e.g. nucleic acids, peptides etc.

As used herein, a “biological sample” refers to a sample including tissues, cells, organs, biological fluids, polypeptides, nucleic acids, or other biological substances. In some embodiments a biological sample can further include preservatives. In some embodiments, a sample can be obtained from a subject. In some embodiments a sample can be a diagnostic sample obtained from a subject. By way of non-limiting example, a sample can be a gamete, sperm, eggs, an embryo, a zygote, chondrocytes, red blood cells, blood, portions or fractions of blood, hepatic cells, fibroblasts, stem cells, cord blood cells, adult stem cells, induced pluripotent stem cells, autologous cells, autologous stem cells, bone marrow cells, hematopoietic cells, hematopoietic stem cells, somatic cells, germ line cells, differentiated cells, somatic stem cells, embryonic stem cells, serum, plasma, sputum, cerebrospinal fluid, urine, tears, alveolar isolates, pleural fluid, pericardial fluid, cyst fluid, tumor tissue, a biopsy, saliva, an aspirate, or combinations thereof. In some embodiments, a sample can be obtained by resection, biopsy, or egg retrieval.

The term “chimeric antigen receptor” or “CAR” as used herein refers to an antigen-binding domain that is fused to an intracellular signaling domain capable of activating or stimulating an immune cell, and in certain embodiments, the CAR also comprises a transmembrane domain. In certain embodiments the CAR's extracellular antigen-binding domain is composed of a single chain variable fragment (scFv) derived from fusing the variable heavy and light regions of a murine or humanized monoclonal antibody. Alternatively, scFvs may be used that are derived from Fab's (instead of from an antibody, e.g., obtained from Fab libraries). In various embodiments, the scFv is fused to the transmembrane domain and then to the intracellular signaling domain. “First-generation” CARs include those that solely provide CD3ζ signals upon antigen binding, “Second-generation” CARs include those that provide both co-stimulation (e.g., CD28 or CD137) and activation (CD3ζ). “Third-generation” CARs include those that provide multiple co-stimulation (e.g. CD28 and CD137) and activation (CD3ζ). A fourth generation of CARs have been described, CAR T cells redirected for cytokine killing (TRUCKS) where the vector containing the CAR construct possesses a cytokine cassette. When the CAR is ligated, the

CAR T cell deposits a pro-inflammatory cytokine into the tumor lesion. A CAR-T cell is a T cell that expresses a chimeric antigen receptor. The phrase “chimeric antigen receptor (CAR),” as used herein and generally used in the art, refers to a recombinant fusion protein that has an antigen-specific extracellular domain coupled to an intracellular domain that directs the cell to perform a specialized function upon binding of an antigen to the extracellular domain. The terms “artificial T-cell receptor,” “chimeric T-cell receptor,” and “chimeric immunoreceptor” may each be used interchangeably herein with the term “chimeric antigen receptor.”

As used herein, the terms “cell,” “cell line,” and “cell culture” may be used interchangeably. All of these terms also include their progeny, which are any and all subsequent generations. It is understood that all progeny may not be identical due to deliberate or inadvertent mutations. The “cells” can be prokaryotic or eukaryotic and encompass all species, e.g. mammals, fish, birds, reptiles, insects, fungi, bacterial and the like. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

“Cryopreserved cells” or “cryopreserved tissues” are cells or tissues that have been preserved by cooling to a sub-zero temperature. Cryopreserved cells include eukaryotic and prokaryotic cells., including pluripotent stem cells, immune cells, blood cells, transformed cells, transfected cells, CAR-T cells, TIL cells, NK cells or any type of cell useful for transfusing or transplanting into a subject, or for diagnostic and research purposes. Cryopreserved cells and tissues include, for example, animal, insect, bird, fish, reptile and plant cells or tissues.

As used herein, the phrase “cryopreservative composition” refers to a chemical or a chemical solution which facilitates the process of cryopreservation by reducing the injury of cells and tissues during freezing and thawing. The cryopreservative compositions protect cells and tissues from damage associated with storage at sub-zero temperature and/or freezing, e.g., cell membrane damage due to ice crystal formation. The term “preservative” media or composition refers to a chemical or a chemical solution which allows the long-term storage and/or culturing of cells at varying temperatures, including room temperature, refrigeration, typical cell-culturing temperatures, freezing temperatures and the like. The compositions of the present disclosure are thus cryoprotective, cryopreservative, preservative, or combinations thereof.

As used herein, the term “immune cells” generally includes white blood cells (leukocytes) which are derived from hematopoietic stem cells (HSC) produced in the bone marrow “Immune cells” includes, e.g., lymphocytes (T cells, B cells, natural killer (NK) cells) and myeloid-derived cells (neutrophil, eosinophil, basophil, monocyte, macrophage, dendritic cells). Among the sub-types and subpopulations of T cells and/or of CD4+ and/or of CD8+ T cells are naive T (T_{N}) cells, effector T cells (T_{EFF}), memory T cells and sub-types thereof, such as stem cell memory T (T_{SCMX} central memory T (T_{CM} effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as T_{H}1 cells, T_{H}2 cells, T_{H}3 cells, T_{H}17 cells, T_{H}9 cells, T_{H}22 cells, follicular helper T cells, alpha/beta T cells, and delta/gamma T cells.

The term “immune effector cell,” as used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NK-T) cells, mast cells, and myeloic-derived phagocytes “Immune effector function or immune effector response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

The term “organ” as used herein refers to a structure of bodily tissue in a subject, e.g., a mammalian subject such as a human, wherein the tissue structure as a whole is specialized to perform a particular bodily function. Organs which are transplanted within the meaning of the present invention include for example, but without limitation, cornea, skin, heart, lung, kidney, pancreas, liver, spleen. In some embodiments, the term “organ” also encompasses decellularized and recellularized organs, as well as engineered and artificial organs and tissues, including engineered organs (e.g., tissue engineered constructs), engineered organs comprising a bioscaffold, tissues, organ slices and partial organs.

“Pharmaceutical agent,” also referred to as a “drug,” or “therapeutic agent” is used herein to refer to an agent that is administered to a subject to treat a disease, disorder, or other clinically recognized condition that is harmful to the subject, or for prophylactic purposes, and has a clinically significant effect on the body to treat or prevent the disease, disorder, or condition. Therapeutic agents include, without limitation, agents listed in the United States Pharmacopeia (USP), Goodman and Gilman's The Pharmacological Basis of Therapeutics, 12^{th} Ed., McGraw Hill, 2001; Katzung, B. (ed.) Basic and Clinical Pharmacology, McGraw-Hill/Appleton & Lange; 8^{th} edition (Sep. 21, 2000); Physician's Desk Reference (Thomson Publishing), and/or The Merck Manual of Diagnosis and Therapy, 18^{th} ed. (2006), or the 19^{th} ed (2011), Robert S. Porter, MD., Editor-in-chief and Justin L. Kaplan, MD., Senior Assistant Editor (eds.), Merck Publishing Group, or, in the case of animals, The Merck Veterinary Manual, 10^{th} ed., Cynthia M. Kahn, B. A., M. A. (ed.), Merck Publishing Group, 2010.

“Polyamino acids” are synthetic polymers made up of many repeating units of an amino acid. Examples include: poly-L-lysine, poly-D-lysine, poly-L-ornithine, etc. The term “amino acid” is taken to include the stereoisomeric forms, for example D and L forms, of amino acids, e.g., alanine, β-alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine, γ-aminobutyrate, NE-acetyllysine, Nδ-acetylornithine, Nγ-acetyldiaminobutyrate and Na-acetyldiaminobutyrate. A “carboxylated polyamino acid” includes any polyamino acid, such as polylysine, polyarginine, polyglutamine, etc., which has a repeating unit that has both amino and carboxyl groups, wherein at least a portion of the amino groups of the polyamino acid are partially blocked by being carboxylated (or acetylated) with carboxylic acid anhydride(s). This blockage is done by the carboxylation of the amino groups to the degrees greater than 50%, and ranging from about 50-99%, in embodiments about 52-90%, in other embodiments from about 55-75%, in still other embodiments about 57-67%, and in still other embodiments about 60%. About 50% of the amino groups would be blocked by being reacted with 52-53 mol % of anhydrous carboxylic acid on basis of molar amount of the amino groups in the polyamino acid. In a normal reaction condition, 90-95% of the amino groups would be blocked when reacted with 100 mol % anhydrous carboxylic acid. Suitable carboxylic acid anhydrides useful in carboxylating polyamino acids include, without limitation, acetic anhydride, citric anhydride, succinic anhydride, glutaric anhydride, malic anhydride, fumaric anhydride and maleic anhydride. Among these, succinic anhydride and acetic anhydride are particularly useful.

As used herein, the term “polysaccharide” refers to chains of mono- or di-saccharide units bound together by glycosidic linkages. They range in structure from linear to highly branched. Some non-limiting examples include starch, glycogen, cellulose, chitin, chitosan, xylan, arabinoxylan, mannan, fucoidan, galactomannan, callose, laminarin, chrysolaminarin, amylopectin, dextran, dextrins, maltodextrins, hyaluronic acid, inulin, oligofructose, polydextrose, polysucrose, pullanan, etc. In embodiments, the preservative or cryopreservative compositions may include at least one polysaccharide comprising dextran, polysucrose, hyaluronic acid. In particular embodiments, the preservative or cryopreservative compositions may include dextran. Dextrans are polysaccharides with molecular weights ≥1000 Dalton, which have a linear backbone of a-linked D-glucopyranosyl repeating units. Three classes of dextrans can be differentiated by their structural features: Class 1 dextrans contain the α(1→6)-linked D-glucopyranosyl backbone modified with small side chains of D-glucose branches with α(1→2), α(1→3), and α(1→4)-linkage. The class 1 dextrans vary in their molecular weight, spatial arrangement, type and degree of branching, and length of branch chains, depending on the microbial producing strains and cultivation conditions. Isomaltose and isomaltotriose are oligosaccharides with the class 1 dextran backbone structure. Class 2 dextrans (alternans) contain a backbone structure of alternating α(1→3) and α(1→6)-linked D-glucopyranosyl units with α(1→3)-linked branches. Class 3 dextrans (mutans) have a backbone structure of consecutive α(1→3)-linked D-glucopyranosyl units with α(1→6)-linked branches. One and two-dimensional NMR spectroscopy techniques have been utilized for the structural analysis of dextrans.

As used herein, the term “preservative compositions” include compositions embodied herein, wherein the biological sample is stored over short e.g. 1 day and over extended periods of time e.g. weeks, months or years at temperatures greater than 0° C., e.g. room or ambient temperatures.

As used herein, the term “saccharide” refers to any carbohydrate comprising monosaccharides (e.g., glucose, ribose, fructose, galactose, etc.), disaccharides (e.g., sucrose, lactose, maltose, cellobiose, trehalose, dextran e.g. dextran-40, melibiose, etc.), oligosaccharides (e.g., raffinose, stachyose, amylose, etc.), and polysaccharides (e.g., starch, glycogen, cellulose, chitin, xylan, arabinoxylan, mannan, fucoidan, galactomannan, callose, laminarin, chrysolaminarin, amylopectin, dextran, dextrins, maltodextrins, inulin, oligofructose, polydextrose, etc.). The term encompasses simple carbohydrates, as well as complex carbohydrates. Indeed, it is not intended that the present invention be limited to any particular saccharide, as various saccharides and forms of saccharides find use in the present invention.

The term “viability” as used herein refers to the state of a cell or a tissue. The cells or tissues may have undergone multiple freeze-thaw cycles in the compositions embodied herein. Viability of cells is also easily determined, for example, immunostaining, dye exclusion, metabolic tests etc. The term “viability” also refers to the state of, a tissue or an organ's survival capability, e.g., capable of survival after transplantation into a recipient. Viability can be used as a measure of the entire organ's survival or a part of the organ, or the viability of cells within the organ. The term “viability” also includes reference to cells, cell cultures, tissues, etc.

It is to be understood that compounds embodied herein that have varying molecular weights are included. For example, dextran −4, −150 etc. Polysucrose 20, −1000 etc.

### Preservative and Cryopreservative Compositions

Cryopreservation involves the storage of biological samples, including cells, tissues, and organs, at sub-zero temperatures at which biological activity effectively ceases. This allows storage of biological samples with minimal degradation of the sample and/or long-term storage of biological samples. Cryopreservation can be performed in a variety of different manners. For example, cryopreservation can be performed at a slower rate, referred to herein as “slow-rate cryopreservation,” wherein the decrease in temperature of the biological sample to sub-zero temperatures is typically performed over minutes, hours, days, etc. As another example, cryopreservation can be performed at a faster rate of cryopreservation, referred to herein as “fast-rate cryopreservation” which includes for example, vitrification and/or ultra-rapid freezing, wherein the decrease in temperature of the biological sample to sub-zero temperatures is typically performed in seconds or fractions of a second, such as milliseconds and at temperatures significantly lower than the temperatures associated with slow-rate cryopreservation. In embodiments, the slow-rate cryopreservation process may occur at temperatures ranging from 0° C. to −100° C. whereas fast-rate cryopreservation processes may occur at temperatures lowers than −100° C.

The cryopreservative compositions described herein may be adopted for use in any type of cryopreservation method, including for example slow-rate cryopreservation, or fast-rate cryopreservation including vitrification, and/or ultra-rapid freezing. These compositions can also be used in long-term storage and/or culturing of cells without the need for freezing. Accordingly, long term storage, lasting years and without freezing, can include maintaining the cells at temperatures typical for storing or culturing. For example, from 0° C. up to 37° C., 40° C. etc., depending on the cell-type.

In embodiments, the compositions may be combined in a physiological solution, such as saline and dextrose, as well as biological media, e.g., Dulbecco's Modified Eagle Medium (DMEM), Dulbecco's Modified Eagle Medium: Nutrient Mixture F-12 (DMEM/F12), F10 Nutrient Mixture, Ham's F12 Nutrient Mixture, Media 199, MEM, Minimum Essential Media (MEM), RPMI Medium 1640 (RPMI-1640), Opti-MEM I Reduced Serum Media, Iscove's Modified Dulbecco's Medium (IMDM) Eagle's Minimal Essential Medium (EMEM), X-VIVO, water, saline, dextrose, and combinations thereof.

In certain embodiments, a preservative or cryopreservative composition comprises a polyamino acid, an organic amphoteric agent, a saccharide or combinations thereof.

In certain embodiments, the organic amphoteric agent is ectoine or derivatives thereof. In certain embodiments, a derivative of ectoine comprises: acetylhydroxectoine, hydroxyectoine, homoectoin, stearoylhydroxyectoine, myristylectoin, or combinations thereof. In certain embodiments, the polyamino acid is poly-L-lysine.

In certain embodiments, the saccharide comprises: a monosaccharide, disaccharide, oligosaccharide, polysaccharide or combinations thereof. In certain embodiments, the saccharide comprises: dextran, α-D-glucopyranosyl-(1→1)-α-D-glucopyranoside (trehalose) or a combination thereof.

In certain embodiments the preservative or cryopreservative composition further comprises one or more other compounds comprising: polysucrose, polyamines, DHMICA (4,5-dihydro-2-methylimidazole-4-carboxylate), cDPG (cyclic 2,3-diphosphoglycerate, potassium salt), DGP (diglycerol phosphate), polyethylene glycol (PEG), glycoin, firoin, succinic anhydride, sodium hydroxide or combinations thereof. In certain embodiments, the compound is polysucrose.

In other embodiments, a preservative or cryopreservative composition comprises ectoine or derivatives thereof, a polyamino acid, a saccharide or combinations thereof. In certain embodiments, the cryopreservative composition further comprises one or more of: polysucrose, DHMICA (4,5-dihydro-2-methylimidazole-4-carboxylate), cDPG (cyclic 2,3-diphosphoglycerate, potassium salt), DGP (diglycerol phosphate), polyethylene glycol (PEG), glycoin, firoin, succinic anhydride, sodium hydroxide or combinations thereof.

In another embodiment, the preservative or cryopreservative composition comprises: poly-L-lysine, ectoine or derivatives thereof, dextran or combinations thereof. In certain embodiments, the preservative or cryopreservative composition comprises: ectoine or derivatives thereof, poly-L-lysine, trehalose or combinations thereof. In certain embodiments, the preservative or cryopreservative composition comprises: ectoine or derivatives thereof, poly-L-lysine, dextran or combinations thereof. In certain embodiments, the preservative or cryopreservative composition comprises: ectoine or derivatives thereof, poly-L-lysine, dextran, polysucrose or combinations thereof. In certain embodiments, the preservative or cryopreservative composition comprises: ectoine or derivatives thereof, poly-L-lysine, trehalose, polysucrose or combinations thereof. In certain embodiments, the preservative or cryopreservative composition comprises: ectoine or derivatives thereof, poly-L-lysine, dextran, trehalose, polysucrose or combinations thereof.

In another embodiment, a composition comprises poly-L-lysine, succinic anhydride, a hydroxide, cell-culture medium or combinations thereof. In certain embodiments, the composition further comprises: ectoine or derivatives thereof, trehalose or combinations thereof. In certain embodiments, the preservative or cryopreservative composition comprises: ectoine or derivatives thereof, dextran or combinations thereof. In certain embodiments, the preservative or cryopreservative composition comprises: ectoine or derivatives thereof, dextran, polysucrose or combinations thereof. In certain embodiments, the preservative or cryopreservative composition comprises: ectoine or derivatives thereof, trehalose, polysucrose or combinations thereof. In certain embodiments, the preservative or cryopreservative composition comprises: ectoine or derivatives thereof, dextran, trehalose, polysucrose or combinations thereof.

In another embodiment, a composition comprises a carboxylated-polyamino acid, ectoine or derivatives thereof, and a polysaccharide. In embodiments, the carboxylated polyamino acid may be derived from a polylysine. Polylysine is intended to include ε-poly-L-lysine or ε-poly-D-lysine or α-poly-L-lysine. The polylysine may include an average molecular weight of about 1,000-20,000 Daltons, and particularly between about 1,000-10,000 Daltons. In certain embodiments, the saccharide comprises: dextran, α-D-glucopyranosyl-(1→1)-α-D-glucopyranoside (trehalose) or a combination thereof. In certain embodiments, the composition further comprises polysucrose, polyamines, DHMICA (4,5-dihydro-2-methylimidazole-4-carboxylate), cDPG (cyclic 2,3-diphosphoglycerate, potassium salt), DGP (diglycerol phosphate), polyethylene glycol (PEG), glycoin, firoin, succinic anhydride, sodium hydroxide or combinations thereof.

In yet another embodiment, a composition comprises a polyamino acid, ectoine or derivatives thereof, dextran, α-D-glucopyranosyl-(1→1)-α-D-glucopyranoside (trehalose) polysucrose, polyamines, DHMICA (4,5-dihydro-2-methylimidazole-4-carboxylate), cDPG (cyclic 2,3-diphosphoglycerate, potassium salt), DGP (diglycerol phosphate), polyethylene glycol (PEG), glycoin, firoin, succinic anhydride, sodium hydroxide or combinations thereof. In certain embodiments, a derivative of ectoine comprises: acetylhydroxectoine, hydroxyectoine, homoectoin, stearoylhydroxyectoine, myristylectoin, or combinations thereof.

In yet another embodiment, a composition comprises ectoin, hydroxyectoin, glycoin, firoin, firoin-A, cDPG (cyclic 2,3-diphosphoglycerate, potassium salt), DGP (diglycerol phosphate), DIP (di-myo-inositolphosphate), homoectoin, DHMICA (4,5-dihydro-2-methylimidazole-4-carboxylate), acetyl-hydroxyectoin, myristylectoin, stearoylhydroxyectoin, or combinations thereof. These can be combined with one or more other compounds such as dextran, trehalose, polysucrose or combinations thereof.

Table 1 shows examples of possible combinations of the additives in the preservation or cryopreservation compositions. These examples are not to be construed as limiting.

TABLE 1

| L1 | CP FORMULATION |
|---|---|
| L2 | CP FORMULATION + 5% Trehalose |
| L3 | P24 + 5% Trehalose |
| L4 | P24 + 5% Polysucrose 20 |
| L5 | P24 + 5% Polysucrose 1000 |
| L6 | P24 + 5% CM-dextran 4 |
| L7 | P24 + 5% CM-dextran 150 |
| L8 | P24 + 5% Q-dextran 4 |
| L9 | P24 + 5% Q-dextran 150 |
| L10 | P24 + 5% dextran T10 |
| L11 | P24 + 5% dextran T70 |

*P24 comprises ε-poly-l-lysine, Dulbecco's modified eagle's medium, succinic anhydride, and sodium hydroxide.
*CP FORMULATION comprises P24 + 5% dextran + 5% ectoine.

In other embodiments, the preservative or cryopreservative compositions may further include one or more pharmaceutically acceptable excipient or pharmaceutically acceptable agent or is diluted in a pharmaceutically acceptable excipient to obtain the desired ratio of agents in the compositions. A pharmaceutically acceptable excipient, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular formulation desired. Remington's The Science and Practice of Pharmacy, 21^{st} Edition, A. R. Gennaro, (Lippincott, Williams & Wilkins, Baltimore, Md., 2006; incorporated herein by reference) discloses various excipients used in formulating pharmaceutical compositions which excipients are useful in preparing the present preservative or cryopreservative compositions. Except insofar as any conventional excipient is incompatible with a substance or its derivatives, such as by producing any undesirable biological effect or otherwise interacting in a deleterious manner with any other component(s) of the pharmaceutical composition, its use is contemplated to be within the scope of this disclosure.

In certain embodiments, a method of preserving or cryopreserving a biological sample comprises obtaining a biological sample; contacting the biological sample with a preservative or cryopreservative compositions embodied herein.

In certain embodiments, a method of preserving or cryopreserving a cell, the method comprising: adding the cell to the preservative or cryopreservative compositions embodied herein, freezing the composition; storing the frozen composition at a temperature below 0° C.; thawing the composition; removing the cell from the thawed composition; and culturing the cell under conditions effective for the cell to remain viable. In certain embodiments, freezing the composition comprises at least one round of cooling, re-warming, and further cooling.

In certain embodiments, a biological medium comprising a cell or tissue culture medium and a preservative or cryopreservative compositions embodied herein.

In certain embodiments, the cells or any biological sample can be preserved over extended periods of time, e.g. 1 day, 2 days, 3 days, 1 week, 2 week, 3 weeks, one month, two months, one year and so forth at ambient or room temperatures.

In some embodiments, the pharmaceutically acceptable excipient is at least 95%, 96%, 97%, 98%, 99%, or 100% pure. In some embodiments, the excipient is approved for use in humans and for veterinary use. In some embodiments, the excipient is approved for use in humans by the United States Food and Drug Administration. In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

In another embodiment, a composition includes a viscosity enhancer. In certain embodiments, the viscosity enhancer is cellulose or a cellulose derivative. In certain embodiments, the viscosity enhancer is carboxymethylcellulose. In certain embodiments, the viscosity enhancer is methyl cellulose. In certain embodiments, the viscosity enhancer is one or more of ethyl cellulose, hydroxyethylcellulose, hydroxypropylcellulose, hydroxyethyl ethylcellulose, hydroxyethylcellulose, hydroxypropylcellulose, or hydroxybutyl cellulose. Other exemplary viscosity enhancers include synthetic polymers (e.g., acrylamides, acrylates). In certain embodiments, the viscosity enhancer is a wax or fatty alcohol (e.g., cetyl alcohol).

In some embodiments, the preservative or cryopreservative compositions further comprise an aldose, a ketose, an amino sugar, a saccharide (e.g., a disaccharide, a polysaccharide, etc.), or combinations thereof. In some embodiments, the preservative or cryopreservative compositions comprise sucrose, dextrose, glucose, lactose, trehalose, dextran e.g. dextran-40, arabinose, pentose, ribose, xylose, galactose, hexose, idose, monnose, mannose, talose, heptose, fructose, gluconicacid, sorbitol, mannitol, methyl α-glucopyranoside, maltose, isoascorbic acid, ascorbic acid, lactone, sorbose, glucaric acid, erythrose, threose, arabinose, allose, altrose, gulose, erythrulose, ribulose, xylulose, psicose, tagatose, glucuronicacid, gluconic acid, glucaric acid, galacturonic acid, mannuronic acid, glucosamine, galactosamine, neuraminic acid, arabinans, fructans, fucans, galactans, galacturonans, glucans, mannans, xylans, levan, fucoidan, carrageenan, galactocarolose, pectins, pectic acids, amylose, pullulan, glycogen, amylopectin, cellulose, dextran, pustulan, chitin, agarose, keratin, chondroitin, dermatan, hyaluronic acid, alginic acid, xanthin gum, starch, polyethyleneglycol, dimethyl sulfoxide, ethylene glycol, propylene glycol, propylene, glycol, polyvinvyl pyrrolidone, glycerol, polyethylene oxide, polyether, serum, or combinations thereof.

In certain embodiment, a preservative or cryoprotective composition comprises one or more cryoprotective agents. In preferred embodiments, the preservative or cryoprotective agent is non-toxic to the cellular matter under the conditions at which it is used (e.g. at a particular concentration, for a particular exposure time and to cells in a medium of a particular osmolality). A preservative or cryoprotective agent may be cell permeating or non-permeating. Examples of preservative or cryoprotective agents include but are not limited to, dehydrating agents, osmotic agents and vitrification solutes (i.e., solutes that aid in the transformation of a solution to a glass rather than a crystalline solid when exposed to low temperatures). In some embodiments, a preservative or cryoprotective agent can be a naturally-occurring cryoprotective agent such as ectoin and/or hydroxyectoin. Other examples of naturally occurring agents or cryoprotectants include, without limitation, anti-freeze proteins, saccharides, ice nucleating agents, compatible solutes, sugars, polyols, glucose, sucrose, glycerol and the like. These can be isolated from nature, synthesized in the laboratory, or obtained from commercial sources. Natural sources include insects, fish, amphibians, animals, birds and plants. Most notably, Arctic and Antarctic insects, fish and amphibians.

In certain embodiments, the compositions further include one or more therapeutic agents, hormones, growth factors, lipids, cytokines, oligonucleotides, polynucleotides, proteins, polypeptides, peptides, small molecules, chemotherapeutic agents and the like (e.g., polyphenols, fatty alcohols). In certain embodiments, cytokines include lymphokines, monokines interferons interleukins (“ILs”) such as IL-1, IL-1α, IL-2, IL-3, IL-4, IL-5, IL-6, IL-7, IL-8, IL-9, IL-10, IL-11, IL-12, IL-13, IL-15, IL-17A-F, IL-18 to IL-29 (such as IL-23), IL-31, including PROLEUKIN™. rIL-2; a tumor-necrosis factor such as TNF-α or TNF-β, TGF-β1-3; and other polypeptide factors including leukemia inhibitory factor (“LIF”), ciliary neurotrophic factor (“CNTF'), CNTF-like cytokine (”CLC″), cardiotrophin (“CT”), and kit ligand (“KL”). As used herein, the term “chemokine” refers to soluble factors (e.g., cytokines) that have the ability to selectively induce chemotaxis and activation of leukocytes. They also trigger processes of angiogenesis, inflammation, wound healing, and tumorigenesis. Example chemokines include IL-8, a human homolog of murine keratinocyte chemoattractant (KC).

In embodiments, the preservative or cryopreservative compositions embodied herein, allow for extreme cooling and thawing rates, overcome toxicity of high cryoprotectant agent (CPA) concentrations, allow for use of small volumes of biological media and are superior to traditional cryopreservative agents.

It will be appreciated that the thawing rate of cryopreserved cells or tissues, for example, will be influenced by a variety of factors. For example, the volume of the cryopreserved cells, handling time, ambient temperature, the temperature of incubation chambers used, heat transfer properties of the container housing the cells, the volume of the cryosolution added to the cryopreserved cells, and the like may influence thawing rate. It will also be appreciated that cells in a particular sample of cryopreserved cells may not all thaw at the same rate or within the same time period. Methods for thawing cryopreserved cells are well known in the art (See, e.g., Freshney R I, Culture of Animal Cells: A Manual of Basic Technique, 4^{th} Edition, 2000, Wiley-Liss, Inc., Chapter 19).

The cryopreserved cells to be thawed may be in a composition that occupies a volume of about 0.1 ml, 0.5 ml, 1 ml, about 2 ml, about 3 ml, about 4 ml, about 5 ml, about 10 ml, about 20 ml, about 30 ml, about 40 ml, about 50 ml, about 100 ml, about 200 ml, about 300 ml, about 400 ml, about 500 ml, about 1 L, or more. The cryopreserved cells may be in a composition that occupies a volume ranging from about 0.1 ml, 0.5 ml, 1 ml to about 10 ml, from about 10 ml to about 20 ml, from about 20 ml to about 30 ml, from about 30 ml to about 40 ml, from about 40 ml to about 50 ml, from about 50 ml to about 100 ml, from about 100 ml to about 200 ml, from about 200 ml to about 300 ml, from about 300 ml to about 400 ml, from about 400 ml to about 500 ml, or from about 500 ml to about 1 L. The composition including the cells may contain a tissue sample, e.g., a blood sample, a fat sample.

Typically, the step of thawing involves obtaining cryopreserved cells from storage at a temperature of less than about 0° C. (a subzero temperature) and allowing them to thaw to a temperature above 0° C. The step of thawing may involve obtaining the cryopreserved cells from storage at a temperature that ranges from about −205° C. to about −195° C. The step of thawing may involve obtaining the cryopreserved cells from storage at a temperature that ranges from about −80° C. to about −60° C. The step of thawing may involve progressively warming the cryopreserved cells by transferring the cells among incubators each having a warmer temperature range, e.g., to control the rate of thawing. For example, the step of thawing may involve first obtaining cryopreserved cells from storage at a first subzero temperature, e.g., that ranges from about −205° C. to about −195° C., and transferring the cryopreserved cells to a second, typically warmer, yet typically subzero, storage temperature, e.g., to a temperature that ranges from about −80° C. to about −60° C., prior to thawing. Any number of stages, for example, 2, 3, 4, 5, 6, or more stages, is envisioned to control the rate of thawing in this manner The step of thawing may also involve progressively warming the cryopreserved cells by incubating the cells in a temperature controlled chamber, e.g., a water bath, heat block, oven, etc., and progressively warming the chamber, e.g., at a controlled rate, while the cryopreserved cells are present in the chamber.

The step of thawing may involve incubating the cryopreserved cells at a temperature that ranges from about 15° C. to about 30° C. The step of thawing may involve incubating the cryopreserved cells at a temperature that ranges from about 30° C. to about 45° C. Such incubation may be performed by incubating a container housing the cryopreserved cells in temperature controlled incubator, e.g., a temperature controlled water bath, a temperature controlled oven, etc. Other incubation methods will be apparent to the skilled artisan.

The step of thawing may be completed within about 30 seconds, about 1 minute, about 2 minutes, about 3 minutes, about 4 minutes, about 5 minutes, about 10 minutes, about 20 minutes, about 30 minutes, about 40 minutes, about 50 minutes, about 1 hour, or more. The step of thawing may be completed within a range of about 1 minute to about 5 minutes. The step of thawing may be completed within a range of about 5 minutes to about 10 minutes. The step of thawing may be completed within a range of about 10 minutes to about 30 minutes. The step of thawing may be completed within a range of about 30 minutes to about 60 minutes.

The step of thawing may involve warming the cryopreserved cells at a rate of about 1° C. per minute, about 2° C. per minute, about 3° C. per minute, about 4° C. per minute, about 5° C. per minute, about 10° C. per minute, about 20° C. per minute, about 30° C. per minute, about 40° C. per minute, about 50° C. per minute, about 60° C. per minute, about 70° C. per minute, about 80° C. per minute, about 90° C. per minute, about 100° C. per minute, about 200° C. per minute, or more. The step of thawing may involve warming the cryopreserved cells at a rate ranging from about 1° C. per minute to about 5° C. per minute. The step of thawing may involve warming the cryopreserved cells at a rate ranging from about 5° C. per minute to about 25° C. per minute. The step of thawing may involve warming the cryopreserved cells at a rate ranging from about 25° C. per minute to about 50° C. per minute. The step of thawing may involve warming the cryopreserved cells at a rate ranging from about 50° C. per minute to about 100° C. per minute. The step of thawing may involve warming the cryopreserved cells at a rate ranging from about 100° C. per minute to about 200° C. per minute. The rate of thawing may be continuous, e.g., constant rates until cells are completely thawed. The rate of thawing may also be discontinuous, e.g., the rate may be more rapid at some temperature ranges relative to the rate at other temperature ranges during thawing, for example, the rate may be more rapid in the range of about −200° C. to about 0° C. than in the range of about 0° C. to about 45° C. during the thawing.

Although not required or necessary, the cells may be washed at any stage during the cryopreservation process. In certain embodiments, the cells are washed after harvesting. In certain embodiments, the cells are washed after thawing. In certain embodiments, the cells are washed before transplantation. Such washing may minimize the presence of any cellular debris resulting from the cell collection process or the cryopreservation process. The washing of cells may be performed using any known methods in the art. For example, the cells may be washed with normal saline or another suitable wash solution. In certain embodiments, the volume of wash solution used is at least equal to the volume of cells being washed. The washing may involve suspending the cells in the wash solution and then centrifuging the cells to collect the washed cells. In other embodiments, the cells are centrifuged without adding any wash solution, and the cell pellet is resuspended in normal saline or another suitable solution for further use such as transplantation. The step of washing may be performed once or multiple times. In certain embodiments, the wash step may be repeated two, three, four, five, six, seven, or more times. Typically, the wash step is not performed more than two to three times. In certain embodiments, only a single wash is performed.

When freezing cells, the concentration of the cells which are to be cryopreserved may vary depending on a variety of factors, including, for example, the type of cell or tissue, the downstream application, etc. The concentration of certain cell types may be low, e.g., for oocytes the concentration may be as low as about 1-30 cells per ml, or lower. The concentration of cells may be about 10⁰ cells/ml, about 10¹ cells/ml, about 10² cells/ml, about 10³ cells/ml, about 10⁴ cells/ml, about 10⁵ cells/ml, about 10⁶ cells/ml, about 10⁷ cells/ml, about 10⁸ cells/ml, about 10⁹ cells/ml, or more. The concentration of cells may range from about 10⁰ cells/ml to about 10¹⁰ cells/ml, from about 10⁰ cells/ml to about 10¹ cells/ml, from about 10¹ cells/ml to about 10² cells/ml, from about 10² cells/ml to about 10³ cells/ml, from about 10³ cells/ml to about 10⁴ cells/ml, from about 10⁴ cells/ml to about 10⁵ cells/ml, from about 10⁵ cells/ml to about 10⁶ cells/ml, from about 10⁶ cells/ml to about 10⁷ cells/ml, from about 10⁷ cells/ml to about 10⁸ cells/ml, or from about 10⁸ cells/ml to about 10⁹ cells/ml, for example.

The methods and compositions disclosed herein may be used with any cryopreserved cells, typically eukaryotic cells. However, the methods and compositions disclosed herein are also envisioned for use with prokaryotic cells. The methods and compositions disclosed herein are also useful with plant cells, insect cells, etc.

Cells may be primary cells isolated from any tissue or organ (e.g., connective, nervous, muscle, fat or epithelial tissue). The cells may be mesenchymal, ectodermal, or endodermal. Cells may also be present in isolated connective, nervous, muscle, fat or epithelial tissue, e.g., a tissue explant, e.g., an adipose tissue obtained by liposuction. The connective tissue may be, for example, bone, ligament, blood, cartilage, tendon, or adipose tissue. The muscle tissue may be vascular smooth muscle, heart smooth muscle, or skeletal muscle, for example. The epithelial tissue may be of the blood vessels, ducts of submandibular glands, attached gingiva, dorsum of tongue, hard palate, esophagus, pancreas, adrenal glands, pituitary glands, prostate, liver, thyroid, stomach, small intestine, large intestine, rectum, anus, gallbladder, thyroid follicles, ependyma, lymph vessel, skin, sweat gland ducts, mesothelium of body cavities, ovaries, Fallopian tubes, uterus, endometrium, cervix (endocervix), cervix (ectocervix), vagina, labia majora, tubuli recti, rete testis, ductuli efferentes, epididymis, vas deferens, ejaculatory duct, bulbourethral glands, seminal vesicle, oropharynx, larynx, vocal cords, trachea, respiratory bronchioles, cornea, nose, proximal convoluted tubule of kidney, ascending thin limb of kidney, distal convoluted tubule of kidney, collecting duct of kidney, renal pelvis, ureter, urinary bladder, prostatic urethra, membranous urethra, penile urethra, or external urethral orifice, for example.

The cells may be any mammalian cells. The cells may be any human cells. The cells include: lymphocytes, B cells, T cells, cytotoxic T cells, natural killer T cells, regulatory T cells, T helper cells, myeloid cells, granulocytes, basophil granulocytes, eosinophil granulocytes, neutrophil granulocytes, hypersegmented neutrophils, monocytes, macrophages, reticulocytes, platelets, mast cells, thrombocytes, megakaryocytes, dendritic cells, thyroid cells, thyroid epithelial cells, parafollicular cells, parathyroid cells, parathyroid chief cells, oxyphil cells, adrenal cells, chromaffin cells, pineal cells, pinealocytes, glial cells, glioblasts, astrocytes, oligodendrocytes, microglial cells, magnocellular neurosecretory cells, stellate cells, boettcher cells; pituitary cells, gonadotropes, corticotropes, thyrotropes, somatotrope, lactotrophs, pneumocyte, type I pneumocytes, type II pneumocytes, Clara cells; goblet cells, alveolar macrophages, myocardiocytes, pericytes, gastric cells, gastric chief cells, parietal cells, goblet cells, paneth cells, G cells, D cells, ECL cells, I cells, K cells, S cells, enteroendocrine cells, enterochromaffin cells, APUD cell, liver cells, hepatocytes, Kupffer cells, bone cells, osteoblasts, osteocytes, osteoclast, odontoblasts, cementoblasts, ameloblasts, cartilage cells, chondroblasts, chondrocytes, skin cells, hair cells, trichocytes, keratinocytes, melanocytes, nevus cells, muscle cells, myocytes, myoblasts, myotubes, adipocyte, fibroblasts, tendon cells, podocytes, juxtaglomerular cells, intraglomerular mesangial cells, extraglomerular mesangial cells, kidney cells, kidney cells, macula densa cells, spermatozoa, sertoli cells, leydig cells, oocytes, and mixtures thereof.

The cells may also be isolated from a diseased tissue, e.g., a cancer. Accordingly, the cells may be cancer cells. For example, the cells may be isolated or derived from any of the following types of cancers: breast cancer; biliary tract cancer; bladder cancer; brain cancer including glioblastomas and medulloblastomas; cervical cancer; choriocarcinoma; colon cancer; endometrial cancer; esophageal cancer; gastric cancer; hematological neoplasms including acute lymphocytic and myelogenous leukemia; T-cell acute lymphoblastic leukemia/lymphoma; hairy cell leukemia; chronic myelogenous leukemia, multiple myeloma; AIDS-associated leukemias and adult T-cell leukemia/lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms' tumor.

The cells may include cord-blood cells, stem cells, umbilical cells, amniotic cells, embryonic stem cells, adult stem cells, cancer stem cells, progenitor cells, autologous cells, isograft cells, allograft cells, xenograft cells, bone marrow cells or genetically engineered cells. The cells may be induced progenitor cells. The cells may be cells isolated from a subject, e.g., a donor subject, which have been transfected with a stem cell associated gene to induce pluripotency in the cells. The cells may be cells which have been isolated from a subject, transfected with a stem cell associated gene to induce pluripotency, and differentiated along a predetermined cell lineage. The cells may be cells including a vector expressing a desired product. These or any other types of cells may be used for transplantation or administration to a subject in need of therapy.

Cells lines of any of the cells disclosed herein may also be used with the methods disclosed herein.

The present disclosure also provides methods of transplanting cells in a subject. The cells or tissues may be autologous, haplotyped matched, transformed cells, allogeneic, xenogeneic, cells expressing a desired product or combinations thereof. The methods typically involve thawing cryopreserved cells which have been frozen in the cryopreservative compositions embodied herein and transplanting the thawed cells in the subject. The method may involve obtaining the cells from a donor that is not the transplant recipient, e.g., for use as an allograft, isograft, or xenograft. The methods may involve obtaining the cells from the subject who is the transplant recipient for use as an autograft. The methods may involve expanding the cells in vitro prior to transplanting. The cells may be cryopreserved while situated in a tissue. The cells may be isolated from a tissue and then cryopreserved. The cells may be cryopreserved while situated in a tissue and isolated from the tissue following thawing.

The resulting cryocell composition may be further processed before implantation into a subject. For example, the cells may be washed, purified, extracted, expanded, or otherwise treated before implantation into a subject.

The cryopreserved cells may be thawed and seeded in a scaffold material that allows for attachment of cells and facilitates production of an engineered tissue. In one embodiment, the scaffold is formed of synthetic or natural polymers, although other materials such as hydroxyapatite, silicone, and other inorganic materials can be used. The scaffold may be biodegradable or non-degradable. Representative synthetic non-biodegradable polymers include ethylene vinyl acetate and polymethacrylate. Representative biodegradable polymers include polyhydroxyacids such as polylactic acid and polyglycolic acid, polyanhydrides, polyorthoesters, and copolymers thereof. Natural polymers include collagen, hyaluronic acid, and albumin Hydrogels are also suitable. Other hydrogel materials include calcium alginate and certain other polymers that can form ionic hydrogels that are malleable and can be used to encapsulate cells.

The scaffolds may be used to produce new tissue, such as vascular tissue, bone, cartilage, fat, muscle, tendons, and ligaments. The scaffold is typically seeded with the cells; the cells are cultured; and then the scaffold implanted. Applications include the repair and/or replacement of organs or tissues, such as blood vessels, cartilage, joint linings, tendons, or ligaments, or the creation of tissue for use as “bulking agents”, which are typically used to block openings or lumens, or to shift adjacent tissue, as in treatment of reflux.

Besides adipocytes, fat tissue has been found to be a source of stem cells (Gimble et al., “Adipose-Derived Stem Cells for Regenerative Medicine” Circulation Research 100:1249-1260, 2007; incorporated herein by reference). Therefore, compositions embodied herein, are useful in stabilizing and preventing damage to stem cells or other cells derived from fat tissue following cryopreservation. In certain embodiments, the compositions are useful in the transplantation of adult stem cells. In certain embodiments, the compositions are useful in the transplantation of fibroblasts.

The preservative or cryopreserved cells may be used for any appropriate downstream application, e.g., research, tissue culture, drug discovery, biologics production, etc. The cells may be used for microscopy, e.g., in combination with immunostaining, in situ hybridization, etc. The cells may be used for functional studies such as gene knockdown or overexpression studies. The cells may be used to study various molecular pathways, e.g., cell cycle, cell signaling, gene regulatory, etc. The cells may be separated by flow cytometry. The cells may be used to create cell lines. The cells may be used for fractionation studies, e.g., to purify proteins or molecules from different cellular compartments. The cells may be used for studying different disease pathways, e.g., cancer. The cells may be transplanted into an animal model, e.g., to study tumor growth. The cells may be used for gene, e.g., mRNA or miRNA, profiling studies. The karyotype or genotype of the cells may be evaluated. The cells may be used for isolation of various biomolecules, e.g., antibodies, proteins, RNA, DNA, ligands, etc.

The cells may be used for automated microscopy for high-content screening, e.g., for lead identification and compound characterization. The cells may be used for the evaluation, e.g., by screening, e.g., high-throughput screening, of compounds, e.g., small-molecules, siRNAs, peptides, etc., for a desired activity, e.g., inhibition of cell growth, modulation of a particular biochemical pathway, modulation of the expression of a certain gene, binding to a target, etc.

The cells may be used in a biopharmaceutical context for the production and isolation of therapeutic molecules, e.g., antibodies, enzymes, etc. The cells may be shipped, e.g., on dry ice in the presence of a polymer, e.g., a polyether, to a customer, collaborator, etc. The cells may be evaluated for contamination, e.g., bacterial, mycoplasmal, viral, etc. The uses disclosed herein are not intended to be limiting and a variety of other uses for the cryopreserved cells are also envisioned and will be apparent to the skilled artisan.

In other embodiments, the preservative or cryopreservative compositions may be used for the preservation or cryopreservation of organs, or for the transport of organs under temperatures suitable for the maintenance of viability of the organ for use in organ transplants and organ donor programs. For the cryopreservation of organs, the organ may be perfused with the cryopreservative compositions and frozen under conditions which preserve the viability of the organ. Procedures for thawing the organs for transplantation are known to those of skill in the art.

The present disclosure also provides kits that include one or more containers filled with agents suitable for formulating the cryopreservative compositions described herein, the containers being enclosed in a single package. For example, the kit may include a first container with a polyamino acid therein, a second container with at least one organic amphoteric agent therein, a third container with a polysaccharide therein, a third container with polysucrose therein. The agents may be in a form ready for mixing or can be premixed, or in concentrated form whereby the user dilutes the concentrated form to predetermined specifications. In some embodiments, the polyamino acid is carboxylated polylysine. In some embodiments, the organic amphoteric agent is ectoine and/or hydroxyectoine. In some embodiments, the organic amphoteric agent includes ectoine, hydroxyectoine, ectoine derivatives, hydroxyectoine derivatives, analogs, variants or combinations thereof. In some embodiments, the polysaccharide is dextran. The kit may also contain one or more diluents, for example, pharmaceutically acceptable excipients, distilled water, saline, biological media, etc.

The kit may also contain instructions for diluting or mixing the agents. The instructions may also include information regarding the contacting of the biological sample with the composition for freezing. Instructions may also include thawing the cryopreserved cells. Such instructions may also include information relating to administration of cells, tissues etc. that had been cryopreserved and thawed.

The kit can also include a notice typically in a form prescribed by a government agency regulating the manufacture, use, or sale of medical devices and/or pharmaceuticals, whereby the notice is reflective of approval by the agency of the compositions, for human or veterinary administration in tissue transplantation.

The kit may include a device or receptacle for preparation of the composition. The device may be, e.g., a measuring or mixing device.

The kit may also optionally include a device for administering the composition of the present disclosure. Exemplary devices include specialized syringes, needles, and catheters that are compatible with a variety of laryngoscope designs.

### EXAMPLES

### Example 1: Composition for High Post-Thaw Viability for Mesenchymal Stem Cells (MSCs) and T Cells

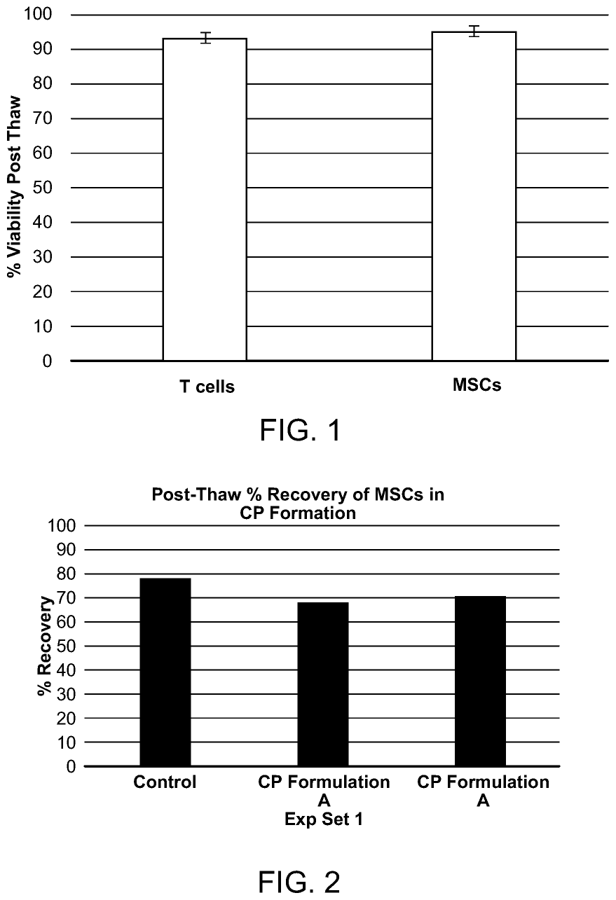

Human bone marrow-derived MSCs and a T cell line that were frozen in CP FORMULATION demonstrate high viability post-thaw (FIG. 1). Post-thaw viability of cells cryopreserved in CP FORMULATION. Cells were frozen at a concentration of 10⁵-10⁶ cells/mL. Cells were stored at −80° C. for 24 hours and then transferred to liquid nitrogen (<-135° C.). After storage in liquid nitrogen for at least 3 days, cells were thawed, and post-thaw viability was assessed. Cells were allowed to recover for at least an additional two days and no changes in cell morphology were observed.

CP FORMULATION is a DMSO-free, serum-free, xeno-free cryopreservation media for long-term preservation of cells in liquid nitrogen. Human bone marrow-derived MSCs and a T cell line that were frozen in CP FORMULATION demonstrate high viability post-thaw (FIG. 1). Cells were frozen at a concentration of 10⁵-10⁶ cells/mL. Cells were stored at −80° C. for 24 hours and then transferred to liquid nitrogen (<−135° C.). After storage in liquid nitrogen for at least 3 days, cells were thawed, and post-thaw viability was assessed. Cells were allowed to recover for at least an additional two days and no changes in cell morphology were observed (FIG. 1).

TABLE 1

| L1 | CP FORMULATION |
|---|---|
| L2 | CP FORMULATION + 5% Trehalose |
| L3 | P24 + 5% Trehalose |
| L4 | P24 + 5% Polysucrose 20 |
| L5 | P24 + 5% Polysucrose 1000 |
| L6 | P24 + 5% CM-dextran 4 |
| L7 | P24 + 5% CM-dextran 150 |
| L8 | P24 + 5% Q-dextran 4 |
| L9 | P24 + 5% Q-dextran 150 |
| L10 | P24 + 5% dextran T10 |
| L11 | P24 + 5% dextran T70 |

*P24 comprises ε-poly-l-lysine, Dulbecco's modified eagle's medium, succinic anhydride, and sodium hydroxide.
*CP FORMULATION comprises P24 + 5% dextran + 5% ectoine.

### Testing Performance of Different Productions of CP FORMULATION.

Four different developmental formulations of CP FORMULATION were made and cell culture testing was completed to evaluate the performance of the cryoprotectant.

Human bone marrow-derived mesenchymal stem cells were frozen in four CP FORMULATION samples as follows:

| | |
|---|---|
| CP FORMULATION | A, B |
| | Run #2, #3 |

Cells were grown in DMEM+10% Fetal Bovine Serum (FBS)+1% Glutamax. General protocols for freezing adherent cells were followed. Cells were detached using 0.25% Trypsin-EDTA. All cell counts were performed using the Trypan Blue Cell Viability Assay using a 1:2 dilution. Detached cells were spun down at 1000 rpms for 5 minutes and resuspended in 1 ml of the cryoprotectant. Testing was divided into two sets. The first test was conducted on CP FORMULATION A and B and cells were frozen at an initial concentration of 5.03×10⁵. The second test was conducted on CP FORMULATION Run #2 and Run #3 at an initial concentration of 4.30×10⁵. The experimental cryoprotectants were measured against a control consisting of 90% PBS and 10% DMSO. Cells were stored in −80° C. for 24 hours and moved to liquid nitrogen. Cells were thawed after one week in liquid nitrogen storage and post-thaw viability was assessed. Cells were re-plated and allowed to grow for 72 hours to observe cell morphology post-thaw.

Percent viability was calculated as:

% viability=(live cells recovered post-thaw)/((total cells recovered post-thaw (live+dead))×100

Percent recovery was calculated as:

% recovery =live cells recovered post-thaw/initial cells seeded×100.

TABLE 2
Experimental set 1 CP FORMULATION post-thaw viability and
recovery from and initial concentration of 5.03 × 10⁵.

| | Final Cell | | |
|---|---|---|---|
| | Concentration | % Via- | % Re- |
| | (cells/mL) | bility | covery |
| Control | 3.13 × 10⁵ | 98% | 78% |
| CP FORMULATION | 3.93 × 10⁵ | 97% | 68% |
| A | | | |
| CP FORMULATION | 4.00 × 10⁵ | 94% | 61% |
| B | | | |

TABLE 3
Experimental Set 1 CP FORMULATION post-thaw average cell counts.

| | | | | | | Average |
|---|---|---|---|---|---|---|
| Cell Counts | | Avg Q1 | Avg Q2 | Avg Q3 | Avg Q4 | (Q1:Q4)/4 |
| Control | Live | 16 | 28 | 14 | 24 | 21 |
| | Dead | 0 | 1 | 0 | 1 | 1 |
| CP | Live | 18 | 18 | 16 | 20 | 18 |
| FORMULATION A | Dead | 0 | 2 | 3 | 2 | 2 |
| CP | Live | 15 | 17 | 13 | 21 | 16 |
| FORMULATION B | Dead | 1 | 2 | 1 | 2 | 2 |

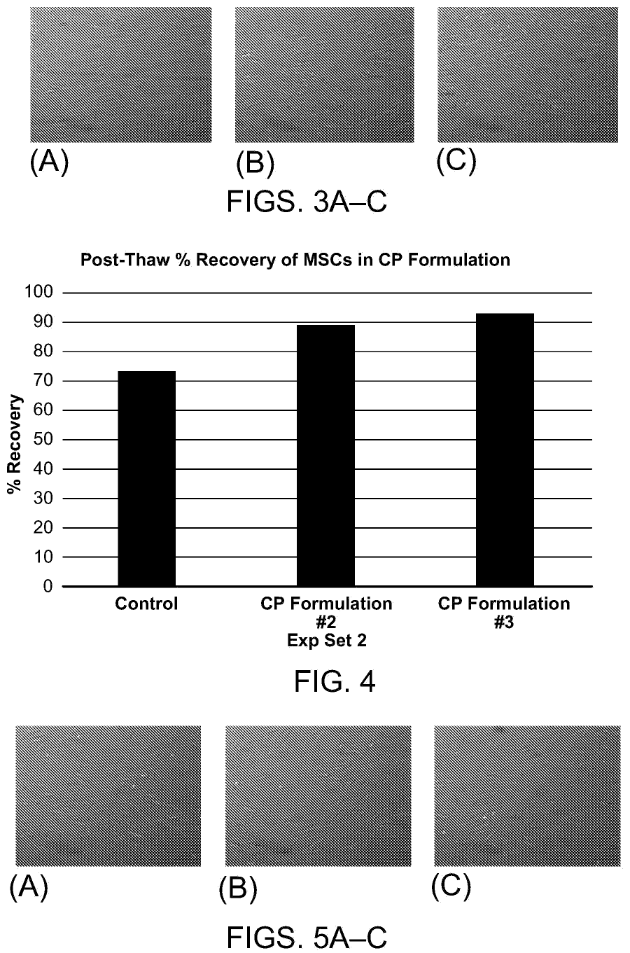

FIG. 2 shows a comparison of post-thaw % recovery of MCS. FIGS. 3A-3C show the post-thaw MSC morphology 72 h after plating.

TABLE 4
Experimental Set 2 CP FORMULATION post-thaw viability and
recovery from and initial concentration of 4.30 × 10⁵.

| | Final Cell | | |
|---|---|---|---|
| | Concentration | % Via- | % Re- |
| | (cells/mL) | bility | covery |
| Control | 3.13 × 10⁵ | 98% | 73% |
| CP FORMULATION | 3.93 × 10⁵ | 97% | 89% |
| #2 | | | |
| CP FORMULATION | 4.00 × 10⁵ | 94% | 93% |
| #3 | | | |

TABLE 5
Experimental Set 2 CP FORMULATION post-thaw average cell counts.

| | | | | | | Average |
|---|---|---|---|---|---|---|
| Cell Counts | | Avg Q1 | Avg Q2 | Avg Q3 | Avg Q4 | (Q1:Q4)/4 |
| Control | Live | 10 | 14 | 16 | 22 | 16 |
| | Dead | 0 | 0 | 0 | 1 | 0 |
| CP FORMULATION #2 | Live | 20 | 19 | 20 | 16 | 19 |
| | Dead | 1 | 0 | 1 | 1 | 1 |
| CP FORMULATION #3 | Live | 17 | 20 | 18 | 25 | 20 |
| | Dead | 1 | 1 | 1 | 2 | 1 |

### Example 3. Cryopreservation of Jurkat Cells with CP FORMULATION

CP FORMULATION is a promising cryoprotectant and has shown its effectiveness in cryopreserving various cell types. The purpose of this study was to evaluate the performance of cryopreserving T cells (Jurkat) with CP FORMULATION.

### Methods

Jurkat clone E6-1 (ATCC® TIB-152™) cells were cryopreserved in three development lots of CP FORMULATION.

The cryoprotectants were tested against an industry standard of 10% DMSO+90% FBS. Jurkat cells were grown initially in RPMI 1040+10% PBS in a T-75 cell culture flask.

All cell counts were performed using the Trypan Blue Cell Viability assay. Cells were frozen at an initial concentration of 1.5×10⁶ cells/ml. Cells were stored in −80° C. for 24 h and then moved to liquid nitrogen.

Cells were thawed after 72 h in liquid nitrogen storage and post-thaw viability was assessed. Cells were resuspended to a total volume of 5 ml with growth media. 1 ml aliquots were taken from the cell suspensions and cell counts were calculated based on the volume of 5 ml. the 1 ml aliquots were transferred back into the cell suspension and the volume was raised to a final total volume 8 ml. Cells were re-plated in a T-25 cell culture flask and allowed to grow for a total of 96 h to observe cell morphology post-thaw.

Percent viability was calculated as:

% viability=(live cells recovered post-thaw)/((total cells recovered post-thaw (live+dead))×100

Percent recovery was calculated as:

% recovery =live cells recovered post-thaw/initial cells seeded×100.

TABLE 6
Post Thaw Cell Counts

| | Avg Total Cell | | | |
|---|---|---|---|---|
| | Concentration | Avg Total | % Via- | % Re- |
| | (cells/mL) | Cell Count | bility | covery |
| Control | 2.45 × 10⁵ | 1.23 × 10⁶ | 92% | 82% |
| CP FORMULATION | 2.67 × 10⁵ | 1.33 × 10⁶ | 92% | 89% |
| #1 | | | | |
| CP FORMULATION | 2.62 × 10⁵ | 1.31 × 10⁶ | 93% | 87% |
| #2 | | | | |
| CP FORMULATION | 3.22 × 10⁵ | 1.61 × 10⁶ | 95% | 107% |
| #3 | | | | |

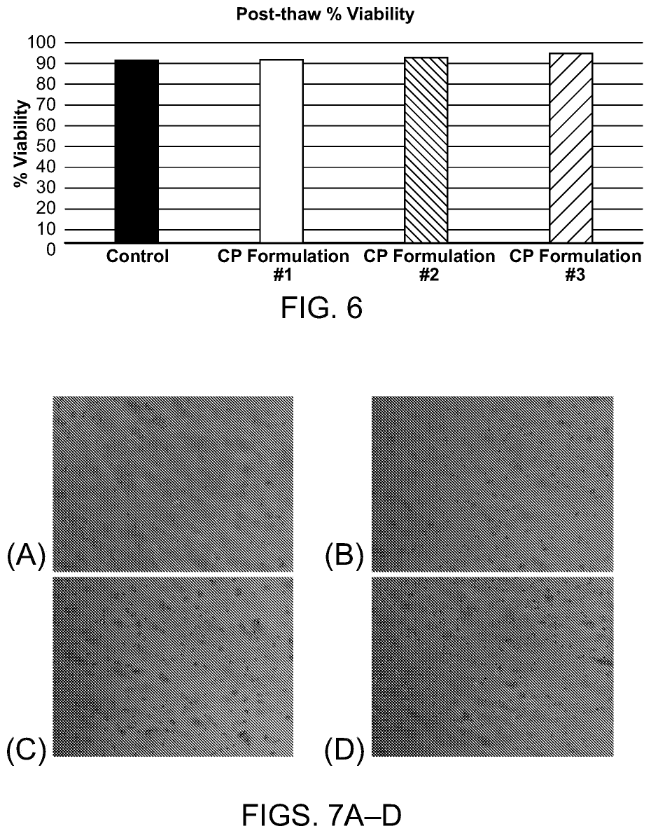

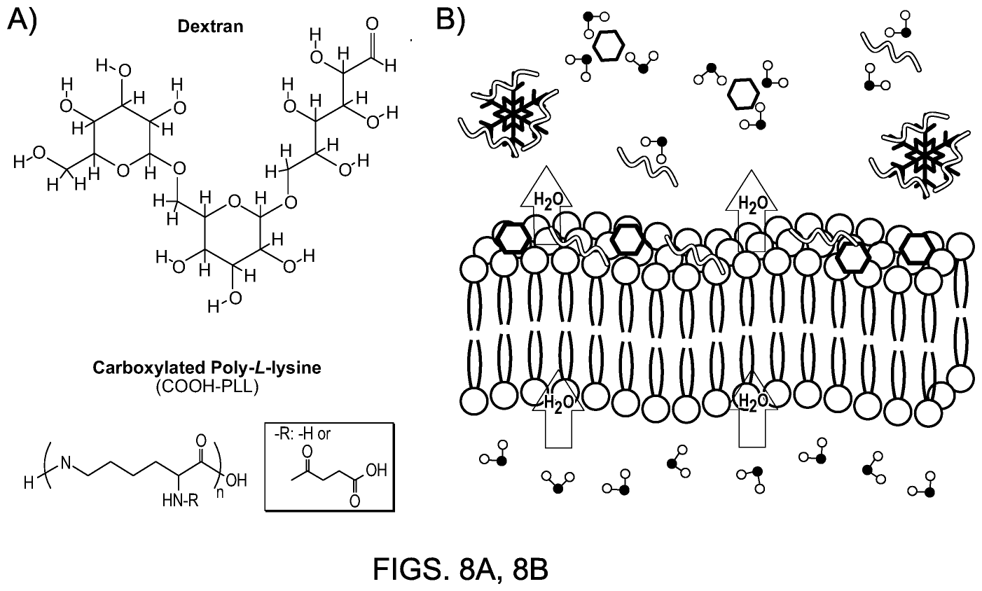

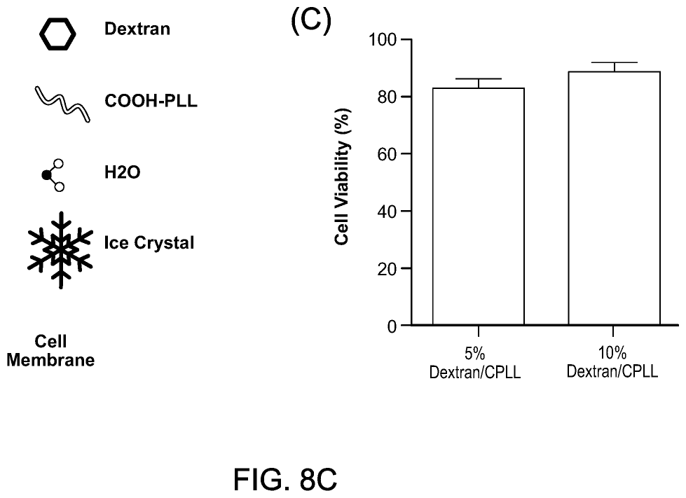

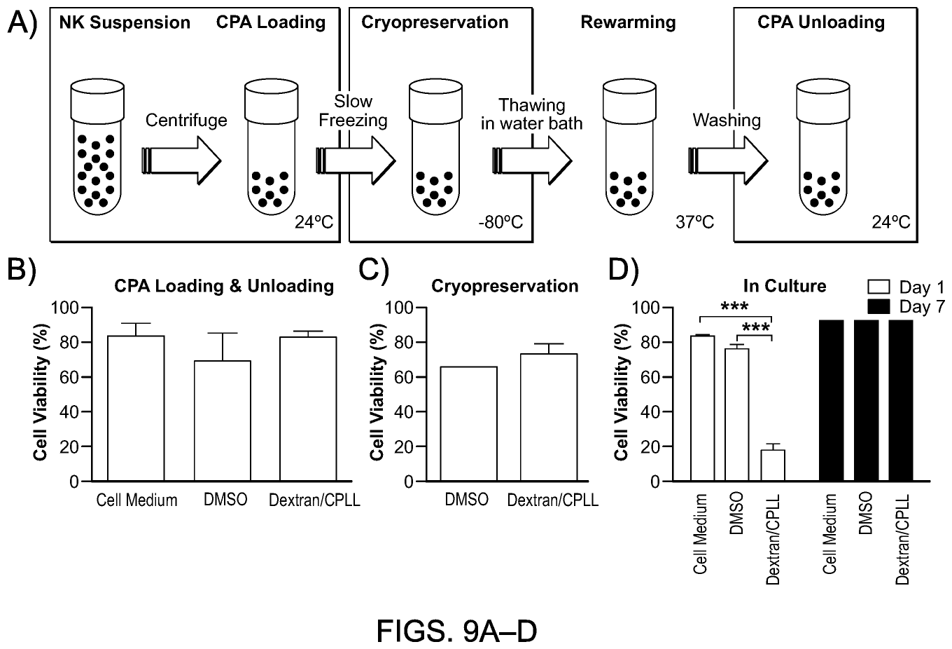

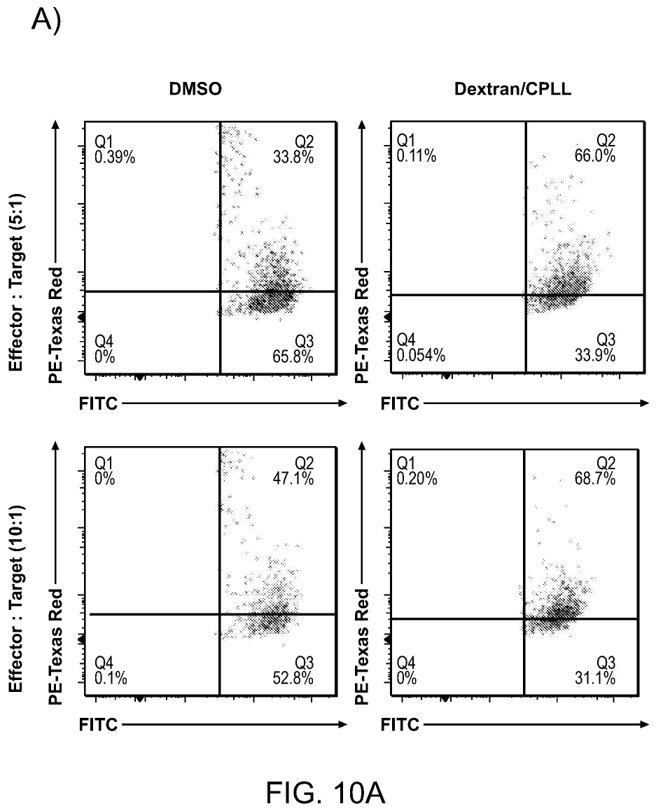

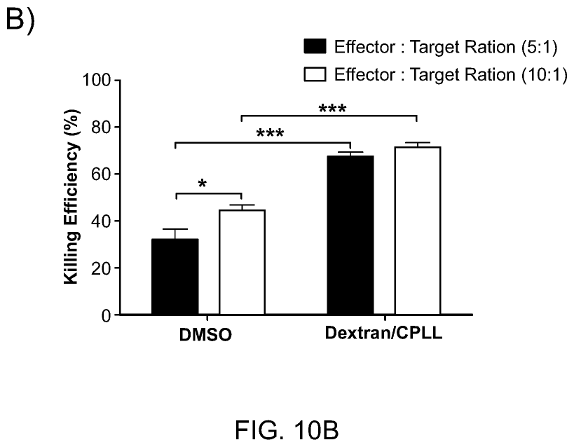

All tested CP FORMULATION samples yielded comparable post-thaw viability and recovery. Jurkat cells in CP FORMULATION also show comparable post-thaw morphology to the control. The results of the bioassay suggest that CP FORMULATION is a strong candidate as a DMSO-free cryopreservation agent for T cells. FIG. 6 is a graph showing the post-thaw cell counts. FIGS. 7A-7D show the comparison of post-thaw morphology of (FIG. 7A) DMSO+PBS; (FIG. 7B) CP FORMULATION Run #1; (FIG. 7C) CP FORMULATION Run #2; (FIG. 7D) CP FORMULATION Run #3.

### Discussion

This bioassay was carried out to evaluate the performance of CP FORMULATION as a cryopreservation agent (CPA) for T Cells. Jurkat cells were used in the bioassay as they are a line of T lymphocytes. Results measured post-thaw viabilities above 90% for all 3 sets of CP FORMULATION. Post-thaw recovery of cells from all 3 sets of CP FORMULATION also showed comparable results. Cells frozen in CP FORMULATION Run #3 have a calculated average recovery slightly above 100%. This is most likely due to a higher than reported concentration of cells frozen for CP FORMULATION Run #3, which also led to a higher calculated total. cell concentration. This can be seen in FIG. 4 with CP FORMULATION Run #3 having a heavier cell density compared to the others.

One complication was encountered involving the incubator during the post-thaw proliferation of Jurkat cells. 48h after plating, there was an unexpected depletion of CO₂ levels in the tank due to a connection error originating from the incubator. Cells were kept inside the incubator and opening of the door was avoided to allow them to grow with the remaining levels of CO₂. The CO₂ tank was replaced 24 h after being found empty and cells were allowed an additional 24 h to continue growth. No morphological difference was observed due the complication in addition to having minimal effects on cell behavior since cells continued to proliferate. One other minor complication that has been reported previously using the same CP FORMULATION trial lots was the appearance of dyed precipitated proteins appearing on the hemocytometer. This has no known effect on performance of CP FORMULATION and is a side effect from Trypan Blue, only causing a minor interference when counting cells.

Further studies on the effects CP FORMULATION has on cellular function is still necessary but CP FORMULATION has demonstrated to be an effective alternative to cryopreservation with DMSO and as a serum-free cryopreservative agent for various cell types.

What is claimed:

1. A preservative or cryopreservative composition comprising a polyamino acid, an organic amphoteric agent, a saccharide or combinations thereof.

2. The preservative or cryopreservative composition of claim 1, wherein the organic amphoteric agent is ectoine or derivatives thereof.

3. The preservative or cryopreservation composition of claim 2, wherein a derivative of ectoine comprises: acetylhydroxectoine, hydroxyectoine, homoectoin, stearoylhydroxyectoine, myristylectoin, or combinations thereof.

4. The preservative or cryopreservative composition of claim 1, wherein the saccharide comprises: a monosaccharide, disaccharide, oligosaccharide, polysaccharide or combinations thereof.

5. The preservative or cryopreservative composition of claim 4, wherein the saccharide comprises: dextran, α-D-glucopyranosyl-(1→1)-α-D-glucopyranoside (trehalose) or a combination thereof.

6. The preservative or cryopreservative composition of claim 1, further comprising polysucrose, polyamines, DHMICA (4,5-dihydro-2-methylimidazole-4-carboxylate), cDPG (cyclic 2,3-diphosphoglycerate, potassium salt), DGP (diglycerol phosphate), DIP (di-myo-inositolphosphate) polyethylene glycol (PEG), glycoin, firoin, succinic anhydride, sodium hydroxide or combinations thereof.

7. The preservative or cryopreservative composition of claim 6 wherein the polyamine is poly-L-lysine.

8. The preservative or cryopreservative composition of claim 1, optionally comprising one or more pharmaceutically acceptable excipients.

9. The preservative or cryopreservative composition of claim 1, further comprising cell-culture media, saline, water, and combinations thereof.

10. A preservative or cryopreservative composition comprising a polyamino acid, ectoine or derivatives thereof, a saccharide or combinations thereof.

11. The preservative or cryopreservative composition of claim 10, wherein a derivative of ectoine comprises: acetylhydroxectoine, hydroxyectoine, homoectoin, stearoylhydroxyectoine, myristylectoin, or combinations thereof.

12. The preservative or cryopreservative composition of claim 10, wherein the saccharide comprises: dextran, α-D-glucopyranosyl-(1→1)-α-D-glucopyranoside (trehalose) or a combination thereof.

13. The preservative or cryopreservative composition of claim 10, further comprising polysucrose, polyamines, DHMICA (4,5-dihydro-2-methylimidazole-4-carboxylate), cDPG (cyclic 2,3-diphosphoglycerate, potassium salt), DGP (diglycerol phosphate), polyethylene glycol (PEG), glycoin, firoin, succinic anhydride, sodium hydroxide or combinations thereof.

14. The preservative or cryopreservative composition of claim 10 wherein the polyamine is poly-L-lysine.

15. The preservative or cryopreservative composition of any one of claims 10 to 14, wherein the composition comprises: poly-L-lysine, ectoine or derivatives thereof, dextran or combinations thereof.

16. The preservative or cryopreservative composition of any one of claims 10 to 14, wherein the composition comprises: ectoine or derivatives thereof, poly-L-lysine, trehalose or combinations thereof.

17. The preservative or cryopreservative composition of any one of claims 10 to 14, wherein the composition comprises: ectoine or derivatives thereof, poly-L-lysine, dextran or combinations thereof.

18. A composition comprising poly-L-lysine, succinic anhydride, a hydroxide, cell-culture medium or combinations thereof.

19. The composition of claim 18, further comprising ectoine or derivatives thereof.

20. The composition of claim 18 or 19, further comprising dextran.

21. The composition of any one of claims 18-20, further comprising polysucrose.

22. The composition of any one of claims 18-21, further comprising α-D-glucopyranosyl-(1→1)-α-D-glucopyranoside (trehalose).

23. The composition of any one of claims 18-22, further comprising DHMICA (4,5-dihydro-2-methylimidazole-4-carboxylate), cDPG (cyclic 2,3-diphosphoglycerate, potassium salt), DGP (diglycerol phosphate), polyethylene glycol (PEG), glycoin, firoin or combinations thereof.

24. A composition comprising a carboxylated-polyamino acid, ectoine or derivatives thereof, and a polysaccharide.

25. The composition of claim 24, wherein the saccharide comprises: dextran, α-D-glucopyranosyl-(1→1)-α-D-glucopyranoside (trehalose) or a combination thereof.

26. The composition of claim 24, further comprising polysucrose, polyamines, DHMICA (4,5-dihydro-2-methylimidazole-4-carboxylate), cDPG (cyclic 2,3-diphosphoglycerate, potassium salt), DGP (diglycerol phosphate), polyethylene glycol (PEG), glycoin, firoin, succinic anhydride, sodium hydroxide or combinations thereof.

27. A composition comprising a polyamino acid, ectoine or derivatives thereof, dextran, α-D-glucopyranosyl-(1→1)-α-D-glucopyranoside (trehalose) polysucrose, polyamines, DHMICA (4,5-dihydro-2-methylimidazole-4-carboxylate), cDPG (cyclic 2,3-diphosphoglycerate, potassium salt), DGP (diglycerol phosphate), polyethylene glycol (PEG), glycoin, firoin, succinic anhydride, sodium hydroxide or combinations thereof.

28. The composition of claim 27, wherein a derivative of ectoine comprises:
acetylhydroxectoine, hydroxyectoine, homoectoin, stearoylhydroxyectoine, myristylectoin, or combinations thereof.

29. A kit comprising a polyamino acid, an organic amphoteric agent, a saccharide or combinations thereof.

30. The kit of claim 29, wherein the organic amphoteric agent is ectoine or derivatives thereof.

31. The kit of claim 30, wherein a derivative of ectoine comprises: acetylhydroxectoine, hydroxyectoine, homoectoin, stearoylhydroxyectoine, myristylectoin, or combinations thereof.

32. The kit of claim 29, wherein the saccharide comprises: a monosaccharide, disaccharide, oligosaccharide, polysaccharide or combinations thereof.

33. The kit of claim 32, wherein the saccharide comprises: dextran, α-D-glucopyranosyl-(1→1)-α-D-glucopyranoside (trehalose) or a combination thereof.

34. The kit of claim 29, further comprising: polysucrose, polyamines, DHMICA (4,5-dihydro-2-methylimidazole-4-carboxylate), cDPG (cyclic 2,3-diphosphoglycerate, potassium salt), DGP (diglycerol phosphate), DIP (di-myo-inositolphosphate) polyethylene glycol (PEG), glycoin, firoin, succinic anhydride, sodium hydroxide or combinations thereof.

35. A composition comprising ectoin, hydroxyectoin, glycoin, firoin, firoin-A, cDPG (cyclic 2,3-diphosphoglycerate, potassium salt), DGP (diglycerol phosphate), DIP (di-myo-inositolphosphate), homoectoin, DHMICA (4,5-dihydro-2-methylimidazole-4-carboxylate), acetyl-hydroxyectoin, myristylectoin, stearoylhydroxyectoin, or combinations thereof.

36. The composition of claim 35, further comprising any one or more compositions of any one of claim 1, 10, 18, 24 or 25.

37. A method of preservation or cryopreserving a biological sample comprising: obtaining a biological sample; contacting the biological sample with a preservative or cryopreservative composition of any one of claim 1, 10, 18, 24, 25 or 35.

38. A biological medium comprising a cell or tissue culture medium and a preservative or cryopreservative composition of any one of claim 1, 10, 18, 24, 25 or 35.

39. A method of cryopreserving a cell, the method comprising: adding the cell to the composition of any one of claim 1, 10, 18, 24, 25 or 35; freezing the composition; storing the frozen composition at a temperature below 0° C.; thawing the composition; removing the cell from the thawed composition; and culturing the cell under conditions effective for the cell to remain viable.

40. The method of claim 39, wherein freezing the composition comprises at least one round of cooling, re-warming, and further cooling.
